(19) United States
(12) Patent Application Publication
(10) Pub. No.: US 2006/0254602 A1
(43) Pub. Date: Nov. 16, 2006

(54) Method of removing unwanted deposits from a keratoprosthesis in vivo

(76) Inventor: Dan L. Myer, Cortersville, GA (US)

Correspondence Address: DAN L. MYER, 25 EQUESTRIAN WAY, CARTERSVILLE, GA, 30121, US

(21) Appl. No.: 11/405,252

(22) Filed: Apr. 17, 2006

Related U.S. Application Data
(60) Provisional application No. 60/671,850, filed on Apr. 15, 2005.

(51) Int. Cl. A61F 9/013; A61F 2/14

(52) U.S. Cl. 128/898; 623/5.16

(57) ABSTRACT

A method of applying a solution in vivo made up of chelating agent(s) such as enzymes and/or surfactants in such a composition to the anterior surface of a keratoprosthesis as to remove deposits and unwanted debris from its surface while maintaining the integrity of the keratoprosthesis.

The applicant is claiming priority in Provisional Case No. 60671850, Filed Apr. 15, 2005.

### BACKGROUND OF THE INVENTION

This invention relates to the field of corneal transplantation surgery; and, more particularly, to the method of keeping various deposits from obstructing the visual axis of a keratoprosthesis.

The cornea is a main component in the refraction of light onto the retina where the image is transmitted to the brain. When the cornea is diseased, or becomes cloudy from trauma or infection, it can be replaced. This can be done with a human corneal graft, or an artificial cornea, or keratoprosthesis.

The human cornea is designed to regenerate apart and independent from a keratoprosthesis. The human cornea sheds epithelium, or its skin, much as the skin on the rest of our body. This process enable a constant renewal of the cornea. The keratoprosthesis does not do this at all, thusly requiring a method of removing deposits periodically while maintaining its integrity, as described in this application.

Visual acuity is measured at subsequent intervals, and found to diminish due to the accumulation of various deposits.

A problem associated with a keratoprosthesis is the undesirable deposits of proteins and lipids, et. al., that accumulate on the surface of the device. These deposits roughen the surface and degrade the optical performance of the keratoprosthesis.

U.S. Pat. No. 6,423,093, which the applicant incorporates in its entirety into this application, describes various prosthetic corneal devices and problems that are and have historically been associated with these corneal devices. U.S. Pat. No. 6,689,165, which the applicant incorporates in its entirety into this application, describes the mechanisms by which some proteins and lipids adhere to the surfaces of a keratoprosthesis made of a variety of polymeric materials.

Depending upon the material from which the keratoprosthesis is made, the deposits may even compromise the structural integrity of the device.

Some patients also experience post-operative infections such as giant papillary conjunctivitis.

Instead of waiting for this to happen and treating the effects of such with steroids or other medicinal drops; the cause of such complications can be addressed with an enzyme, or other chelating agent in the form of application or drop instillation, in vivo.

Eventually, the deposits on the keratoprosthesis degrade the performance of the keratoprosthesis so much that it no longer functions at an acceptable level.

The conventional method for correcting the problems caused by these deposits is to remove the prosthesis and replace it with a new one. This approach exposes the patient again to all the risks and the months-long recovery period associated with the original keratoplasty. When a second corneal implantation surgery is performed, the patient is exposed to new risks beyond those associated with the original keratoplasty. After the keratoplasty, the patient's eye is more fragile structurally. Scar tissue in the eye is weaker than pre-keratoplasty tissue. The recovery period after a second corneal implant procedure is typically longer than the original recovery period. The body is also more likely to reject a second keratoprosthesis. All these problems with keratoprostheses interfere with the long-term efficacy of their use in patients who must have a keratoplasty.

Compositions intended to remove the deposits from hard and soft contact lenses are known. U.S. Pat. No. 4,096,870, which the applicant incorporates in its entirety into this application, describes the use of an aqueous slurry of pancreatin to remove deposits from hydrophilic gel-type soft contact lenses. Compositions employing different active ingredients and other methods for removing undesirable deposits from contact lenses are described in U.S. Pat. Nos. 3,910,296; 4,614,549; 4,921,630; 5,370,744; 5,529,678; 5,858,937; 6,008,037; 6,258,591; and 6,280,530; and U.S. Published Patent Application Nos. 2003/0165545 A1 and 2004/0121924 A1; each of which the applicant incorporates in its entirety into this application.

These patents and patent applications uniformly caution against using the cleaning solutions described in vivo. Therefore, a need exists for a method of removing accumulated deposits on the keratoprosthesis in vivo so that another surgery to replace the device is not required.

A study was developed and a series of tests were performed on the sample material of the keratoprosthesis. Various amounts of an enzyme, and for various periods of time, were applied to the material for evaluation.

It was assessed the longer deposits remain on contact lenses, the more difficult it is to remove them. This led to further testing into the ideas of preventative care via patient instillation of such composition.

A formal method of application was constructed by applying the chelating solution to the keratoprosthesis via sterile, sponge-like applicator, then irrigating.

Subsequently, given the absence of corneal tissue, it is feasible to instill chelating solution directly into the ocular area without corneal incidence; thus making a pre-emptive drop feasible.

After acceptable results were established and confirmed, the results were shown to a corneal surgeon for his inference.

The procedure was then tested on a patient with excellent results. The patient's keratoprosthesis was more clear immediately following the surgery. The patient's visual acuity was restored to best correctable within 24 hours of the procedure.

The initial patient's best correctable vision was 20/60—prior to deposits forming on the keratoprosthesis. The vision diminished to 20/200 best correctable prior to the in vivo procedure.

Immediately following the procedure, his visual acuity measured 20/80—2 in the operated eye. After 24 hours, he was 20/60—in the operated eye; his best correctable vision recorded status post artificial keratoplasty.

### BRIEF SUMMARY OF THE INVENTION

The inventor discovered that with an application of an enzyme solution, deposits were effectively removed, and the integrity of the keratoprosthesis was maintained.

Furthermore, it was concluded the visual acuity was restored to its best correctable state as measured following the initial penetrating keratoplasty.

Another objective with acuities taken before and after the procedure, the degree of visual rehabilitation can be established and proven. The initial patient reaction after the procedure proves this in subjective fact.

Another objective with the ease of deposit removal instead of subsequent penetrating keratoplasties, the life of the keratoprosthesis is prolonged exponentially.

Another objective directly applying cleaning solution in liquid form without touching the implant with an applicator is an alternative method of removing the deposits.

Another objective is patient application of solution as a pre-emptive drop would further prolong the life of the keratoprosthesis.

These and other advantages and objectives of the present invention will become apparent from the detailed description and claims that follow.

### BRIEF DESCRIPTION OF THE DRAWINGS

The figures collectively illustrate one exemplary method of applying in vivo such a composition to the anterior surface of a keratoprosthesis.

FIG. 1 illustrates a cross-section of the cornea with undesirable deposits represented by dots on the anterior surface keratoprosthesis. An examination of the patient's eye with a slit lamp and under magnification will reveal whether such deposits are present.

FIG. 2 illustrates a cross-section of the cornea with the cleaning solution and a sterile, sponge-tipped applicator to be used in the removal of the deposits. One suitable applicator is a sponge-tipped applicator sold by Medtronic under the Weck-Cel® trademark. Sterile cotton-tipped swabs are also suitable as are other sterile applicators.

FIG. 3 illustrates a cross-section of the cornea with application of the cleaning solution to the tip of a sterile, sponge-tipped applicator.

FIG. 4 illustrates a cross-section of the cornea with the applicator and cleaning solution being applied to the anterior surface of the keratoprosthesis to remove the deposits (protein, lipid, calcium, et. al.). This method, in particular, for removing deposits is performed with the patient lying on his back under a microscope so that the physician has more control in the application of the cleaning solution. If needed, the physician may swirl the applicator in order the loosen the deposits.

FIG. 5 illustrates a cross-section of the cornea with the initial stages of the removal of the deposits. These steps would be repeated until desired results were achieved.

FIG. 6 illustrates a cross-section of the cornea with the end stages of the removal of the deposits.

FIG. 7 illustrates a cross-section of the cornea with rinsing of the keratoprosthesis using sterile balanced salt solution. Sterile saline solution, et. al., may be used as well.

FIG. 8 illustrates a cross-section of the cornea with a more clear surface, thus providing the patient with improved vision; and, ultimately prolonging the life of the keratoprosthesis.

### DETAILED DESCRIPTION OF THE INVENTION

The applicant has conceived a method of using cleaning solutions containing enzyme or chelating agents that dissolve these deposits in vivo, and to remove such deposits from the surface of a keratoprosthesis. For example, a composition containing pancreatin marketed by Alcon Laboratories under the trademarks OPTI-FREE® SUPRACLENSE® may be used in vivo with great success in removing undesirable deposits from a keratoprosthesis.

Alternatively, the method for removing deposits may be performed while the patient is sitting erect at the slit lamp.

The method is preferably performed while holding open the patient's eye with a speculum or lid retractor so that the cleaning solution may be applied with precise control.

Alternatively, the patient's eyelids may be held open by hand while the cleaning solution is applied.

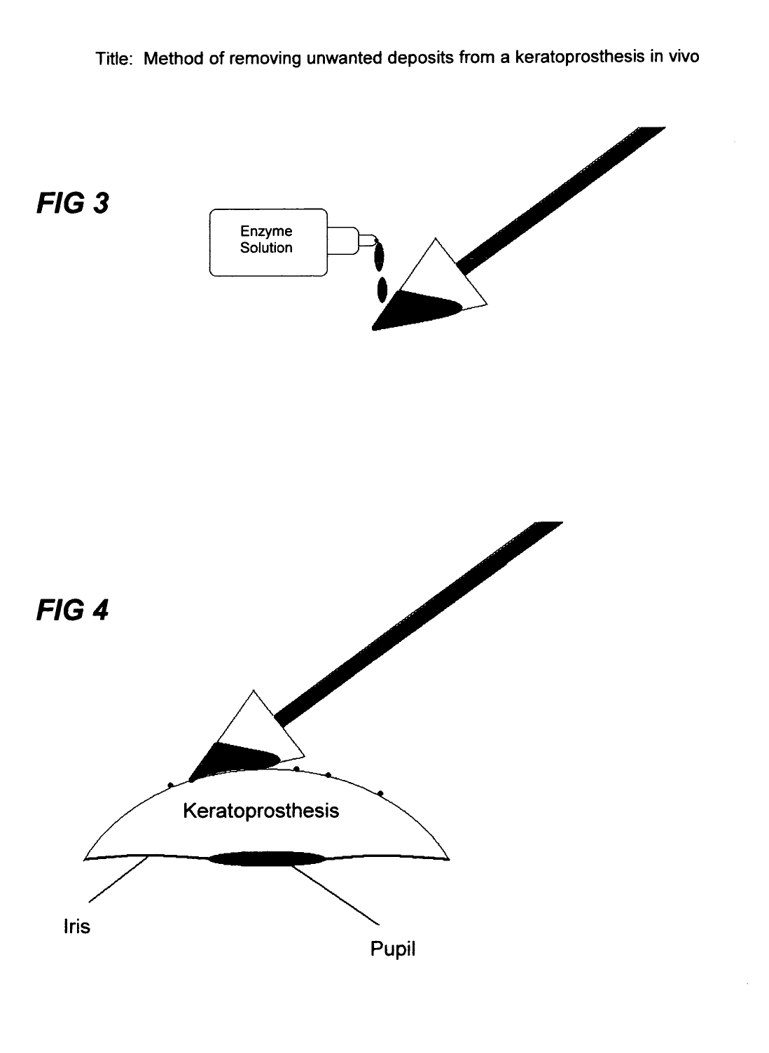

As seen in FIG. 3, approximately 0.5-3 ml of cleaning solution is applied to the applicator tip.

As seen in FIG. 4, the applicator tip, impregnated with cleaning solution, is gently brought into contact with the area on which deposits have been identified on the keratoprosthesis for approximately 15-90 seconds, depending on the density and thickness of the deposits.

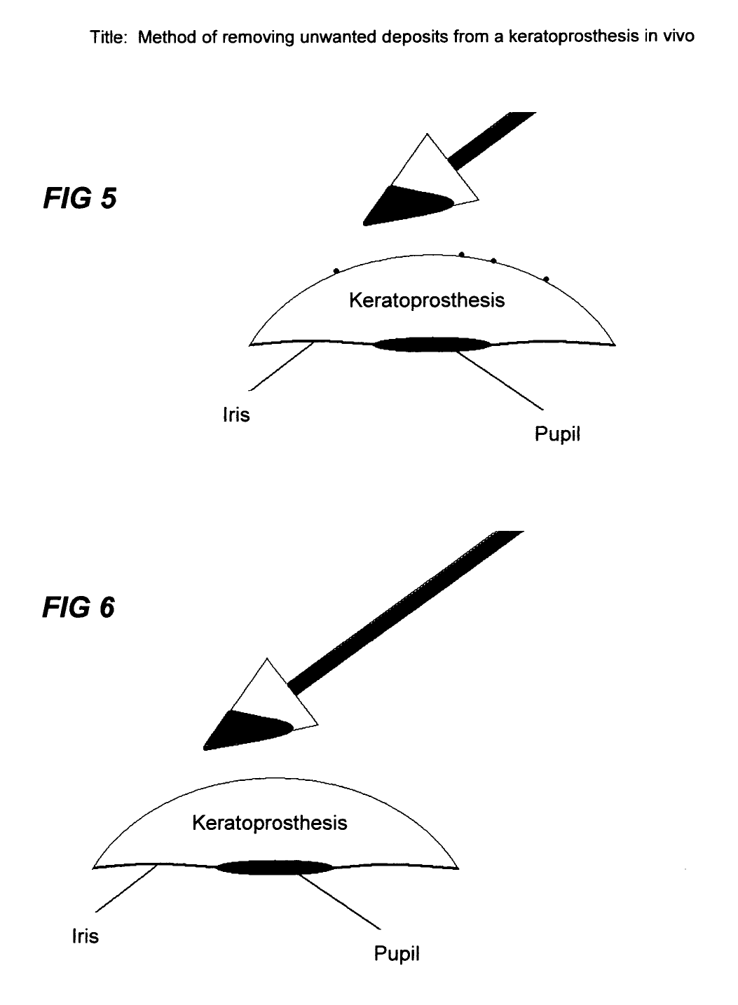

As seen in FIG. 5-6, the combination of the chemical cleaning action of the solution applied and the friction between the applicator and the surface of the implant removes the deposits (protein, lipid, calcium, et. al.).

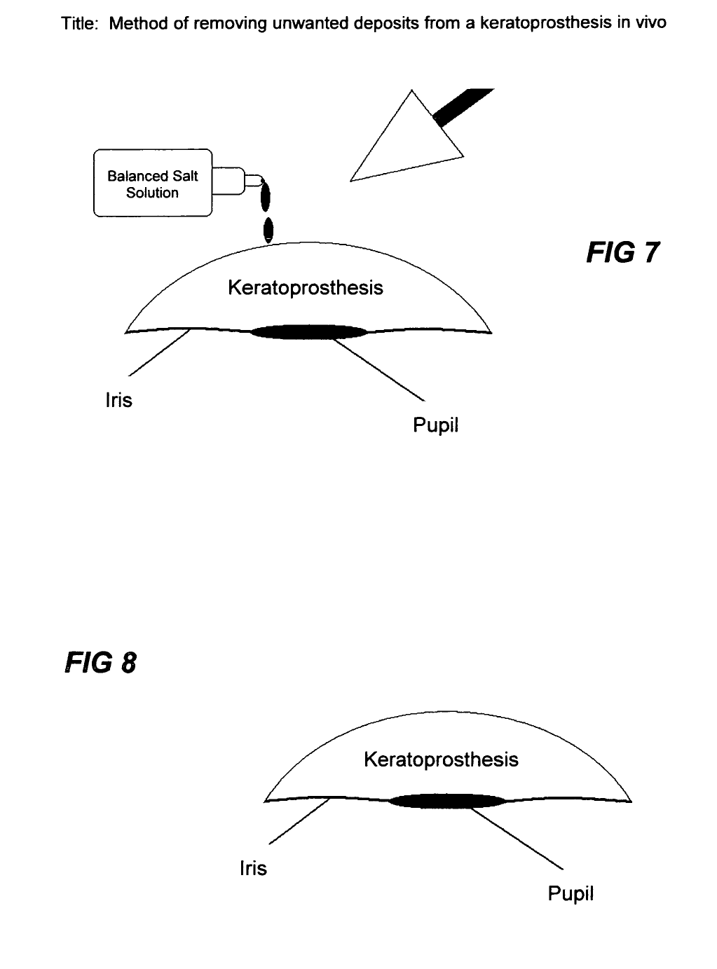

As seen in FIG. 7, the keratoprosthesis is rinsed with balanced salt solution, then evaluated for further removal of deposits until, as in FIG. 8, the surface is clear to an acceptable level.

This description is given for purposes of illustration and explanation. It will be apparent to those skilled in the relevant art that changes and modifications may be made to the invention described above without departing from its scope or spirit.

### BACKGROUND OF THE INVENTION

This invention relates to the field of corneal transplantation surgery; and, more particularly, to the method of keeping various deposits from obstructing the visual axis of a keratoprosthesis.

The cornea is a main component in the refraction of light onto the retina where the image is transmitted to the brain. When the cornea is diseased, or becomes cloudy from trauma or infection, it can be replaced. This can be done with a human corneal graft, or an artificial cornea, or keratoprosthesis.

The human cornea is designed to regenerate apart and independent from a keratoprosthesis. The human cornea sheds epithelium, or its skin, much as the skin on the rest of our body. This process enable a constant renewal of the cornea. The keratoprosthesis does not do this at all, thusly requiring a method of removing deposits periodically while maintaining its integrity, as described in this application.

Visual acuity is measured at subsequent intervals, and found to diminish due to the accumulation of various deposits.

A problem associated with a keratoprosthesis is the undesirable deposits of proteins and lipids, et. al., that accumulate on the surface of the device. These deposits roughen the surface and degrade the optical performance of the keratoprosthesis.

U.S. Pat. No. 6,423,093, which the applicant incorporates in its entirety into this application, describes various prosthetic corneal devices and problems that are and have historically been associated with these corneal devices. U.S. Pat. No. 6,689,165, which the applicant incorporates in its entirety into this application, describes the mechanisms by which some proteins and lipids adhere to the surfaces of a keratoprosthesis made of a variety of polymeric materials.

Depending upon the material from which the keratoprosthesis is made, the deposits may even compromise the structural integrity of the device.

Some patients also experience post-operative infections such as giant papillary conjunctivitis.

Instead of waiting for this to happen and treating the effects of such with steroids or other medicinal drops; the cause of such complications can be addressed with an enzyme, or other chelating agent in the form of application or drop instillation, in vivo.

Eventually, the deposits on the keratoprosthesis degrade the performance of the keratoprosthesis so much that it no longer functions at an acceptable level.

The conventional method for correcting the problems caused by these deposits is to remove the prosthesis and replace it with a new one. This approach exposes the patient again to all the risks and the months-long recovery period associated with the original keratoplasty. When a second corneal implantation surgery is performed, the patient is exposed to new risks beyond those associated with the original keratoplasty. After the keratoplasty, the patient's eye is more fragile structurally. Scar tissue in the eye is weaker than pre-keratoplasty tissue. The recovery period after a second corneal implant procedure is typically longer than the original recovery period. The body is also more likely to reject a second keratoprosthesis. All these problems with keratoprostheses interfere with the long-term efficacy of their use in patients who must have a keratoplasty.

Compositions intended to remove the deposits from hard and soft contact lenses are known. U.S. Pat. No. 4,096,870, which the applicant incorporates in its entirety into this application, describes the use of an aqueous slurry of pancreatin to remove deposits from hydrophilic gel-type soft contact lenses. Compositions employing different active ingredients and other methods for removing undesirable deposits from contact lenses are described in U.S. Pat. Nos. 3,910,296; 4,614,549; 4,921,630; 5,370,744; 5,529,678; 5,858,937; 6,008,037; 6,258,591; and 6,280,530; and U.S. Published Patent Application Nos. 2003/0165545 A1 and 2004/0121924 A1; each of which the applicant incorporates in its entirety into this application.

These patents and patent applications uniformly caution against using the cleaning solutions described in vivo. Therefore, a need exists for a method of removing accumulated deposits on the keratoprosthesis in vivo so that another surgery to replace the device is not required.

A study was developed and a series of tests were performed on the sample material of the keratoprosthesis. Various amounts of an enzyme, and for various periods of time, were applied to the material for evaluation.

It was assessed the longer deposits remain on contact lenses, the more difficult it is to remove them. This led to further testing into the ideas of preventative care via patient instillation of such composition.

A formal method of application was constructed by applying the chelating solution to the keratoprosthesis via sterile, sponge-like applicator, then irrigating.

Subsequently, given the absence of corneal tissue, it is feasible to instill chelating solution directly into the ocular area without corneal incidence; thus making a preemptive drop feasible.

After acceptable results were established and confirmed, the results were shown to a corneal surgeon for his inference.

The procedure was then tested on a patient with excellent results. The patient's keratoprosthesis was more clear immediately following the surgery. The patient's visual acuity was restored to best correctable within 24 hours of the procedure.

The initial patient's best correctable vision was 20/60—prior to deposits forming on the keratoprosthesis. The vision diminished to 20/200 best correctable prior to the in vivo procedure.

Immediately following the procedure, his visual acuity measured 20/80—2 in the operated eye. After 24 hours, he was 20/60—in the operated eye; his best correctable vision recorded status post artificial keratoplasty.

### BRIEF SUMMARY OF THE INVENTION

The inventor discovered that with an application of an enzyme solution, deposits were effectively removed, and the integrity of the keratoprosthesis was maintained.

Furthermore, it was concluded the visual acuity was restored to its best correctable state as measured following the initial penetrating keratoplasty.

Another objective with acuities taken before and after the procedure, the degree of visual rehabilitation can be established and proven. The initial patient reaction after the procedure proves this in subjective fact.

Another objective with the ease of deposit removal instead of subsequent penetrating keratoplasties, the life of the keratoprosthesis is prolonged exponentially.

Another objective directly applying cleaning solution in liquid form without touching the implant with an applicator is an alternative method of removing the deposits.

Another objective is patient application of solution as a preemptive drop would further prolong the life of the keratoprosthesis.

These and other advantages and objectives of the present invention will become apparent from the detailed description and claims that follow.

### BRIEF DESCRIPTION OF THE DRAWINGS

The figures collectively illustrate one exemplary method of applying in vivo such a composition to the anterior surface of a keratoprosthesis.

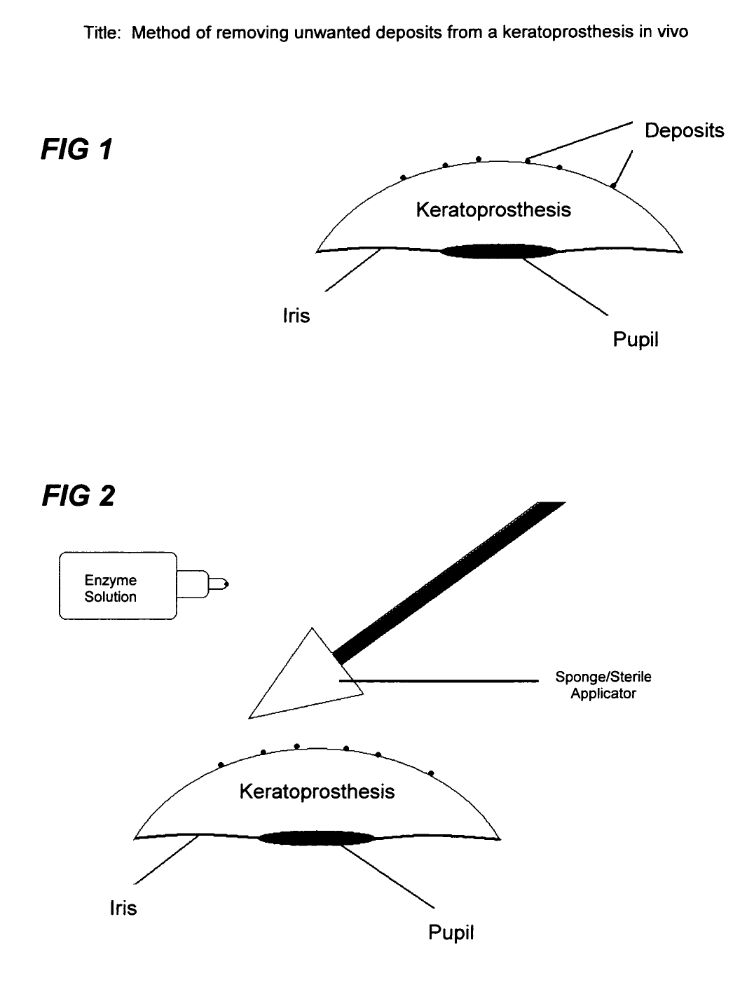

FIG. 1 illustrates a cross-section of the cornea with undesirable deposits represented by dots on the anterior surface keratoprosthesis. An examination of the patient's eye with a slit lamp and under magnification will reveal whether such deposits are present.

FIG. 2 illustrates a cross-section of the cornea with the cleaning solution and a sterile, sponge-tipped applicator to be used in the removal of the deposits. One suitable applicator is a sponge-tipped applicator sold by Medtronic under the Weck-Cel® trademark. Sterile cotton-tipped swabs are also suitable as are other sterile applicators.

FIG. 3 illustrates a cross-section of the cornea with application of the cleaning solution to the tip of a sterile, sponge-tipped applicator.

FIG. 4 illustrates a cross-section of the cornea with the applicator and cleaning solution being applied to the anterior surface of the keratoprosthesis to remove the deposits (protein, lipid, calcium, et. al.). This method, in particular, for removing deposits is performed with the patient lying on his back under a microscope so that the physician has more control in the application of the cleaning solution. If needed, the physician may swirl the applicator in order the loosen the deposits.

FIG. 5 illustrates a cross-section of the cornea with the initial stages of the removal of the deposits. These steps would be repeated until desired results were achieved.

FIG. 6 illustrates a cross-section of the cornea with the end stages of the removal of the deposits.

FIG. 7 illustrates a cross-section of the cornea with rinsing of the keratoprosthesis using sterile balanced salt solution. Sterile saline solution, et. al., may be used as well.

FIG. 8 illustrates a cross-section of the cornea with a more clear surface, thus providing the patient with improved vision; and, ultimately prolonging the life of the keratoprosthesis.

### DETAILED DESCRIPTION OF THE INVENTION

The applicant has conceived a method of using cleaning solutions containing enzyme or chelating agents that dissolve these deposits in vivo, and to remove such deposits from the surface of a keratoprosthesis. For example, a composition containing pancreatin marketed by Alcon Laboratories under the trademarks OPTI-FREE® SUPRACLENS® may be used in vivo with great success in removing undesirable deposits from a keratoprosthesis.

Alternatively, the method for removing deposits may be performed while the patient is sitting erect at the slit lamp.

The method is preferably performed while holding open the patient's eye with a speculum or lid retractor so that the cleaning solution may be applied with precise control.

Alternatively, the patient's eyelids may be held open by hand while the cleaning solution is applied.

As seen in FIG. 3, approximately 0.5-3 ml of cleaning solution is applied to the applicator tip.

As seen in FIG. 4, the applicator tip, impregnated with cleaning solution, is gently brought into contact with the area on which deposits have been identified on the keratoprosthesis for approximately 15-90 seconds, depending on the density and thickness of the deposits.

As seen in FIG. 5-6, the combination of the chemical cleaning action of the solution applied and the friction between the applicator and the surface of the implant removes the deposits (protein, lipid, calcium, et. al.).

As seen in FIG. 7, the keratoprosthesis is rinsed with balanced salt solution, then evaluated for further removal of deposits until, as in FIG. 8, the surface is clear to an acceptable level.

This description is given for purposes of illustration and explanation. It will be apparent to those skilled in the relevant art that changes and modifications may be made to the invention described above without departing from its scope or spirit.

1. This application describes new in vivo methods for removing deposits from the surface of a keratoprosthesis, or artificial corneal implant. More particularly, the application describes methods of evaluating the degree to which deposits (protein, lipid, calcium, epithelium, remnants, et. al.) have compromised the optical, mechanical, and/or biological performance of a keratoprosthesis and applying compositions in vivo that include active, or chelating, agents for removing those deposits.

2. The method of claim 1 wherein irrigating fluid is Balanced Salt Solution.

3. The method of claim 1 wherein irrigating fluid is Sterile Saline Solution.

4. The method of claim 1 wherein irrigating fluid is other sterile fluid combination thereof, or other.

5. The method of claim 1 wherein deposits are protein in nature.

6. The method of claim 1 wherein deposits are lipid in nature.

7. The method of claim 1 wherein deposits are of a calcium nature.

8. The method of claim 1 wherein deposits are epithelial in nature.

9. The method of claim 1 wherein deposits are of other human remnants in nature.

10. The method of claim 1 wherein deposits are of environmental in nature (i.e. airborne debris, pollen, etc.)

11. The method of claim 1 whereas various chelating agents included but not limited to the enzymes in question: Bromelin or Bromelain, Bromolein, Subtilisin, Subtilisin A, Catalase, Ficin, Animal Lipase, Pancreatin, Papain, Pepsin, and/or Trypsin.

12. Method of claim 1 wherein surfactants may be inclusive in the various chelating agents/solutions described and can aid in the removal of deposits.

13. Application of enzymatic agent directly onto keratoprosthesis without applicator, thus resulting in an irrigation and removal of deposits.

14. The method of claim 13 wherein irrigating fluid is Balanced Salt Solution.

15. The method of claim 13 wherein irrigating fluid is Sterile Saline Solution.

16. The method of claim 13 wherein irrigating fluid is other sterile fluid combination thereof, or other.

17. The method of claim 13 wherein deposits are protein in nature.

18. The method of claim 13 wherein deposits are lipid in nature.

19. The method of claim 13 wherein deposits are of a calcium nature.

20. The method of claim 13 wherein deposits are epithelial in nature.

21. The method of claim 13 wherein deposits are of other human remnants in nature.

22. The method of claim 13 wherein deposits are of environmental in nature (i.e. airborne debris, pollen, etc.)

23. The method of claim 13 whereas various chelating agents included but not limited to the enzymes in question: Bromelin or Bromelain, Bromolein, Subtilisin, Subtilisin A, Catalase, Ficin, Animal Lipase, Pancreatin, Papain, Pepsin, and/or Trypsin.

24. Method of claim 13 wherein surfactants may be inclusive in the various chelating agents/solutions described and can aid in the removal of deposits.

25. Introduction of enzymatic agent into the surgical site may be performed via enzyme-soaked applicator. The mechanical manipulation of the applicator combined with the chelating agents may aid in the removal of the deposits.

26. The method of claim 25 may be performed with sterile Weck-Cel® by Medtronic, sterile Q-tip® applicator, or the like.

27. The method of claim 25 wherein irrigating fluid is Balanced Salt Solution.

28. The method of claim 25 wherein irrigating fluid is Sterile Saline Solution.

29. The method of claim 25 wherein irrigating fluid is other sterile fluid combination thereof, or other.

30. The method of claim 25 wherein deposits are protein in nature.

31. The method of claim 25 wherein deposits are lipid in nature.

32. The method of claim 25 wherein deposits are of a calcium nature.

33. The method of claim 25 wherein deposits are epithelial in nature.

34. The method of claim 25 wherein deposits are of other human remnants in nature.

35. The method of claim 25 wherein deposits are of environmental in nature (i.e. airborne debris, pollen, etc.)

36. The method of claim 25 whereas various chelating agents included but not limited to the enzymes in question: Bromelin or Bromelain, Bromolein, Subtilisin, Subtilisin A, Catalase, Ficin, Animal Lipase, Pancreatin, Papain, Pepsin, and/or Trypsin.

37. Method of claim 25 wherein surfactants may be inclusive in the various chelating agents/solutions described and can aid in the prevention/removal of deposits.

38. Periodically applying drops of a cleaning, chelating, and/or enzyme (et. al.) solution, directly to the keratoprosthesis will not only remove accumulated deposits but also help prevent problematic deposits from forming.

39. The method of claim 38 wherein deposits are protein in nature.

40. The method of claim 38 wherein deposits are lipid in nature.

41. The method of claim 38 wherein deposits are of a calcium nature.

42. The method of claim 38 wherein deposits are epithelial in nature.

43. The method of claim 38 wherein deposits are of other human remnants in nature.

44. The method of claim 38 wherein deposits are of environmental in nature (i.e. airborne debris, pollen, etc.)

45. The method of claim 38 whereas various chelating agents included but not limited to the enzymes in question: Bromelin or Bromelain, Bromolein, Subtilisin, Subtilisin A, Catalase, Ficin, Animal Lipase, Pancreatin, Papain, Pepsin, and/or Trypsin.

46. Method of claim 38 wherein surfactants may be inclusive in the various chelating agents/solutions described and can aid in the removal of deposits.
